(19) United States
(12) Patent Application Publication
(10) Pub. No.: US 2009/0227975 A1
(43) Pub. Date: Sep. 10, 2009

(54) TAMPON INCLUDING CROSSLINKED CELLULOSE FIBERS AND IMPROVED SYNTHESIS PROCESSES FOR PRODUCING SAME

(75) Inventors: Eugene Dougherty, JR., Camden-Wyoming, DE (US); Andrew Wilkes, Sandbed Lane Belper (GB)

Correspondence Address: MICHAUD-DUFFY GROUP LLP, 306 INDUSTRIAL PARK ROAD, SUITE 206, MIDDLETOWN, CT, 06457, US

(73) Assignee: Playtex Products, LLC, Shelton, CT (US)

(21) Appl. No.: 12/370,687

(22) Filed: Feb. 13, 2009

Related U.S. Application Data
(60) Provisional application No. 61/029,073, filed on Feb. 15, 2008.

(51) Int. Cl. A61F 13/20; D21C 9/00

(52) U.S. Cl. 604/367; 162/9; 604/385.17

(57) ABSTRACT

A tampon pledget includes crosslinked cellulose fibers having microstructures treated to provide improved absorbency and higher wet strength. The fibers are treated with a crosslinking agent to provide at least one of a molecular weight between crosslinks of from about 10 to 200 and a degree of crystallinity of from about 25% to 75%. The crosslinking agent includes citric acid in 1% by weight. The crosslinking agent may further include sodium hypophosphite in 1% by weight. In another embodiment, the crosslinking agent may be a difunctional agent including a glyoxal or a glyoxal-derived resin. In still another embodiment, the crosslinking agent is a multifunctional agent including a cyclic urea, glyoxal, polyol condensate. The crosslinking agent is added in an amount from about 0.001% to 20% by weight based on a total weight of cellulose fibers to be treated and, preferably, in an amount of about 5% by weight.

### CROSS-REFERENCE TO RELATED APPLICATIONS

This patent application claims priority benefit under 35 U.S.C. §119(e) of copending, U.S. Provisional Patent Application Ser. No. 61/029,073, filed Feb. 15, 2008, the disclosure of this U.S. patent application is incorporated by reference herein in its entirety.

### BACKGROUND OF THE INVENTION

1. Field of the Invention

This invention relates generally to absorbent articles such as catamenial tampons and methods for making such tampons and, more particularly, to tampon pledgets comprised of crosslinked cellulose fibers formed using improved synthetic approaches.

2. Description of the Related Art

A wide variety of configurations of absorbent catamenial tampons are known in the art. Typically, commercially available tampons are made from a tampon pledget that is compressed into a generally cylindrical form having an insertion end and a withdrawal end. A string is generally coupled to the withdrawal end to assist in removing the tampon from the vaginal cavity after use. Before compression, the tampon pledget is typically rolled, spirally wound, folded or otherwise assembled as a rectangular pad of absorbent material.

Many commercially available tampon pledgets are made of cellulose fibers such as rayon. Rayon has many advantages for tampon applications including, for example: it is absorbent; generally recognized as safe and hygienic for use in the human body; raw materials are reasonably low cost; it can be derived from sustainable, natural sources (e.g., eucalyptus trees); and manufacturing processes are well established and commercially viable. Moreover, rayon can be easily blended with other fibers such as, for example, cotton, to tailor properties toward particular applications. However, problems still exist with the use of rayon for tampons. For example, rayon was initially developed as an “artificial silk” and used in apparel, home furnishing and in the manufacture of tires. Rayon was also adapted for use in the feminine care. The inventors have realized, however, that this adaptation did not involve an in-depth effort to modify the attributes of rayon to the special needs of feminine care. For example, it appears that polymeric synthetic routes have not been determined to optimize a cellulosic synthetic fiber to satisfy the unique balance of properties required for feminine care. Rather, improvements of commercial tampons to date have instead focused on design changes and physical process changes seeking to, for example, increase how much or how fast a tampon expands.

One conventional method for forming catamenial tampons includes the use of bulking, crimping and texturing of a continuous filament rayon yarn, wet cross-linking the yarn and twisting or stretching yarn to produce a tampon. Such a forming method is said to provide tampons exhibiting an increase in the volume of water taken up per gram of fiber as well as an increase in wet diameter. Perceived problems in this formation method include the use of formaldehyde as a cross-linking agent; the use of rayon yarn rather than nonwoven materials; and the fact that few, if any, analytical measures, such as molecular weight and extent of crosslinking and crystallinity, were employed to evaluate effectiveness and safety of the formed tampons.

It is also known that more liquid could be held in an absorbent if the stiffness of the fibers is increased by either chemical or physical (e.g., compression) means. Increased stiffness and, in particular, higher wet strength, decreases the tendency of the fiber to draw together and thus maintain greater inter-fiber capillary volumes in which the absorbed fluid could reside. In the case of compressed absorbent materials, the dry modulus and dry resilience must be taken into account. Maximum fluid holding ability in compressed assemblies requires fibers with high wet modulus, coupled with a low modulus and resilience in the dry state. By this method, the desired dry compaction can be achieved under the lowest possible forces of compression, without the excessive forces that lead to permanent setting and fiber damage. On contact with liquid, the fiber transitions from low to high modulus rates. It is generally known that wet crosslinked rayon, a fiber that has the requisite combination of dry and wet state properties, provides a sixty-two percent (62%) increase by measure of volume capacity at compressed bulk densities.

It is also known that crosslinked cellulosic fibers produce absorbent products that wick and redistribute fluid better than non-crosslinked cellulosic fibers due to enhanced wet bulk properties. An inability of wetted cellulosic fibers in absorbent products to further acquire and to distribute liquid to sites remote from liquid intake may be attributed to the loss of fiber bulk associated with liquid absorption. Further, crosslinked cellulosic fibers generally have enhanced wet bulk compared to non-crosslinked fibers. The enhanced bulk is a consequence of the stiffness, twist, and curl imparted to the fiber as a result of the crosslinking. As such, it is generally acknowledged that crosslinked fibers should be incorporated into absorbent products to enhance their bulk as well as speed up the liquid acquisition rates.

It is recognized that synthetic schemes could leverage the above-mentioned findings to provide better and safer synthesis processes for balancing properties of rayon to improve conventional tampon pledgets.

Accordingly, the inventors have discovered that there is a need for an improved tampon pledget formed from crosslinked cellulose fibers and, in particular, for a tampon pledget that is formed from crosslinked rayon that exhibits a desired molecular weight between crosslinks and a balance of order (e.g., crystallinity) and disorder (e.g., amorphous regions) to improve tampon absorbency. The present invention meets this need.

### SUMMARY OF THE INVENTION

The present invention is directed to a tampon pledget including crosslinked cellulose fibers having microstructures treated to provide improved absorbency. The fibers are treated with a crosslinking agent to provide at least one of a molecular weight between crosslinks of from about ten (10) to about two hundred (200) and a degree of crystallinity of from about twenty-five percent (25%) to about seventy-five percent (75%). In one embodiment, the crosslinking agent is comprised of a difunctional crosslinking agent. The difunctional crosslinking agent may include a glyoxal or a glyoxal-derived resin. In one embodiment, the crosslinking agent is comprised of a multifunctional crosslinking agent. The multifunctional crosslinking agent may include a cyclic urea, glyoxal, polyol condensate.

In one embodiment, the crosslinking agent is added in an amount from about one thousandth of one percent (0.001%) to about twenty percent (20%) by weight based on a total weight of cellulose fibers to be treated. In still another embodiment, the crosslinking agent is added in an amount of about five percent (5%) by weight based on the total weight of cellulose fibers.

### BRIEF DESCRIPTION OF THE DRAWINGS

The features and advantages of the present invention will be better understood when the Detailed Description of the Preferred Embodiments given below is considered in conjunction with the figures provided.

FIG. 1 depicts a conventional process for forming viscous rayon fibers.

FIG. 2 depicts a process for forming crosslinked cellulose fibers, in accordance with one embodiment of the present invention.

FIG. 3 illustrates basic cellulose chemistry, as is known in the art.

FIG. 4 depicts a three-dimensional view of a stereochemistry of atoms in cellulose molecule, with an example hydroxyl (—OH) group highlighted as a site for crosslinking and/or hydrogen bonding.

FIG. 5 illustrates molecular weight distributions for various grades of pulp used in rayon manufacture.

FIG. 6 illustrates wet tenacities for various grades of rayon, where the wet tenacity at 5% elongation is typically used to evaluate wet strength in conventional rayon and where the wet tenacity value is higher for rayon made in accordance with the present invention.

FIG. 7 illustrates a method for preparing bags for bagged tampons in accordance with one embodiment of the present invention.

FIG. 8 illustrates a machine set-up for forming tampons in accordance with the present invention.

### DETAILED DESCRIPTION OF THE PREFERRED EMBODIMENTS

In accordance with the present invention, a tampon pledget is formed from crosslinked cellulose fibers such as, for example, rayon. In one aspect of the invention, an overall molecular weight of the crosslinked rayon is adjusted, as is the percent crosslinking and the molecular weight between crosslinks in order to increase the absorbency of the crosslinked rayon and to achieve a balance in dry modulus and wet modulus that leads to better performing tampons.

Tampon performance considerations are addressed by tampon pledgets formed in accordance with the present invention to provide an ability to: (a) absorb viscoelastic fluids like menses more than conventional tampons; (b) absorb menses faster than conventional tampons; (c) conform to the shape and contours of the vagina better to enhance wearing comfort; (d) prevent early bypass failure by expanding rapidly during use to occlude all routes by which fluids could escape the vaginal cavity; (e) exhibit high gram per gram syngyna absorbencies required by agencies such as the Food and Drug Administration (FDA) that regulates tampons; (f) require only a small amount of force to remove the tampon from an applicator; and (g) maintain stability of these aforementioned properties under high temperature and humidity.

As described herein, the present invention has combined and/or adjusted a number of synthetic properties to provide an improved tampon pledget. In one aspect of the present invention, basic cellulosic raw materials used in rayon synthesis, as well as the most common and recognized process for forming rayon, namely the viscous process, were examined. As is generally known, rayon can be produced from almost any cellulosic source. Conventional sources include, for example, pulp from hardwoods, pulp from softwoods, bacterial cellulose, switchgrass, jute, hemp, flax, ramie, and the like. Some of these sources include large percentages of non-cellulosic components, for example, lignin and hemicelluloses, that have few advantages for use as rayon based tampons. Moreover, these raw material sources exhibit significant orientation and crystallinity that detracts from rayon's absorbency properties. Accordingly, it has been discovered that pulp from, for example, eucalyptus trees, contains high proportions of cellulose (e.g., about ninety-eight percent (98%)), are easy to grow in large plantations (e.g., it is thin and fast growing) and thus, are a good source of raw material for providing rayon in accordance with aspects of the present invention.

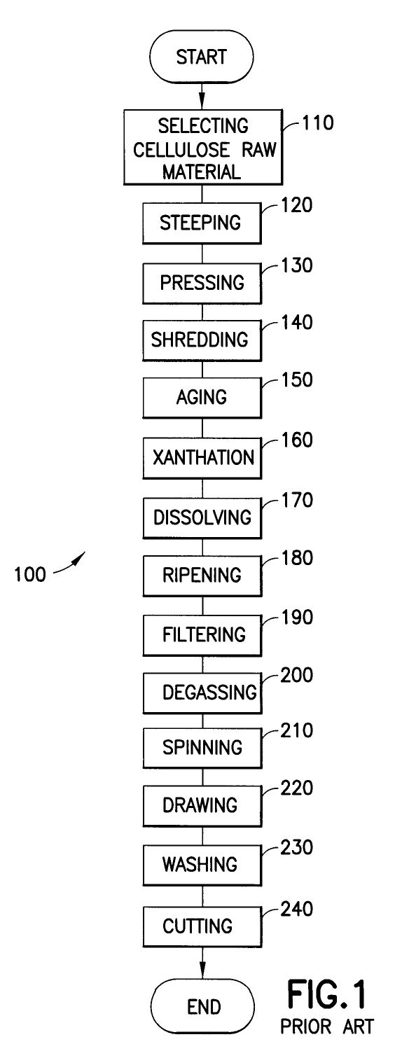

With a raw material source selected, focus was on synthetic routes, as applied to the viscose rayon forming process. As illustrated in FIG. 1, a conventional process 100 of manufacturing viscose rayon includes steps of: selecting, steeping, pressing, shredding, aging, xanthation, dissolving, ripening, filtering, degassing, spinning, drawing, washing, and cutting to provide staple rayon fibers. As noted above, at Block 110, a cellulose raw material is selected. At Block 120, the steeping step includes immersing the cellulose raw material in an aqueous solution of, for example, about seventeen to twenty percent (17-20%) sodium hydroxide (NaOH) at a temperature in the range of about eighteen to twenty-five degrees Celsius (18 to 25° C.) to swell the cellulose fibers and convert the cellulose to alkali cellulose. The alkali cellulose is passed to Block 130 where, in the pressing step, the swollen alkali cellulose is pressed to a wet weight of about two and a half to three (2.5 to 3.0) times its original raw material weight. The pressing is typically performed to provide a preferred ratio of alkali to cellulose. At Block 140, the pressed alkali cellulose is shredded to finely divided particles or “crumbs.” As can be appreciated, shredding the pressed alkali cellulose increases the surface area of the alkali cellulose thus increasing its ability to react in later steps of the viscose forming process. At Block 150, the shredded alkali cellulose is aged under controlled time and temperature conditions to break down the cellulose polymers (e.g., depolymerize the cellulose) to a desired level of polymerization. Typically, the shredded alkali cellulose is aged for about two or three days (about 48 to 72 hours) at temperatures between about eighteen to thirty degrees Celsius (18 to 30° C.). The aging step generally reduces the average molecular weight of the original cellulose raw material by a factor of two to three. Aging and the resulting reduction of the cellulose's molecular weight are performed to provide a viscose solution of desired viscosity and cellulose concentration. The aged alkali cellulose is passed to Block 160 where a xanthation step is performed. At Block 160, the aged alkali cellulose crumbs are added to vats and a liquid carbon disulphide is introduced. The alkali cellulose crumbs react with carbon disulphide under controlled temperatures from about twenty to thirty degrees Celsius (20 to 30° C.) to form cellulose xanthate. At Block 170, the cellulose xanthate is dissolved in a diluted solution of caustic soda (e.g., sodium hydroxide (NaOH)) at temperatures of about fifteen to twenty degrees Celsius (15 to 20° C.) under high-shear mixing conditions to form a viscous solution generally referred to as viscose.

The viscous solution is passed from Block 170 to Block 180, where the viscose is allowed to stand for a period of time to “ripen.” During ripening, two reactions occur, namely, redistribution and loss of xanthate groups. The reversible xanthation reaction allows some of the xanthate groups to revert to cellulosic hydroxyls. Also, carbon disulphide (CS2) is freed. The freed CS2 escapes or reacts with other hydroxyl on other portions of the cellulose chain. In this way, the ordered or crystalline regions are gradually broken down and a more complete solution is achieved. As is generally known, the CS2 that is lost reduces the solubility of the cellulose and facilitates regeneration of the cellulose after it is formed into a filament. At Block 190, the viscose is filtered to remove any undissolved materials. After filtering, the viscose is passed to Block 200 where a degassing step (e.g., vacuum treatment) removes bubbles of air entrapped in the viscose to avoid voids or weak spots that may form in the rayon filaments.

From Block 200, the degassed viscose is passed to Block 210 where an extrusion or spinning step forms viscose rayon filament. At Block 210 the viscose solution is metered through a spinneret into a spin bath containing, for example, sulphuric acid, sodium sulphate, and zinc sulphate. The sulphuric acid acidifies (e.g., decomposes) the sodium cellulose xanthate, the sodium sulphate imparts a high salt content to the bath which is useful in rapid coagulation of viscose, and the zinc sulphate exchanges with the sodium xanthate to form zinc xanthate to cross-link the cellulose molecules. Once the cellulose xanthate (viscose solution) is neutralized and acidified, rapid coagulation of the rayon filaments occurs. At Block 220, in a drawing step, the rayon filaments are stretched while the cellulose chains are relatively mobile. Stretching causes the cellulose chains to lengthen and orient along the fiber axis. As the cellulose chains become more parallel, interchain hydrogen bonds form and give the rayon filaments properties necessary for use as textile fibers (e.g., luster, strength, softness and affinity for dyes). For example, the simultaneous stretching and decomposition of cellulose xanthate slowly regenerates cellulose at a desired tenacity and leads to greater areas of crystallinity within the fiber.

At Block 230, the regenerated rayon is purified by washing to remove salts and other water-soluble impurities. Several conventional washing techniques may be used such as, for example, an initial thoroughly washing, treating with a dilute solution of sodium sulfide to remove sulfur impurities, bleaching to remove discoloration (e.g., an inherit yellowness of the cellulose fibers) and impart an even color, and a final washing. At Block 240, the purified rayon filaments (typically referred to as “tow”) are cut to desired lengths of fiber (typically referred to as “staple” fiber) by, for example, a rotary cutter and the like. The staple rayon fiber is then ready for use in a desired application.

As is generally known, the steps of the above-described viscous rayon forming process 100 can be modified to impart varying characteristics to the rayon fibers. For example, high modulus and high tenacity rayon is made using an Asahi steam explosion process (Asahi Chemical Industry Co. Ltd, Osaka, Japan). In another modified process, the cellulose raw material is complexed with a mixture consisting of cupric oxide and ammonia to provide a cuprammonium rayon. In another modified process, the cellulose raw material produces high tenacity rayon by using N-methyl morpholine N-oxide (NMMO) as a polar solvent or suspension agent (e.g., Tencel or Lyocell rayons). In yet another modified process, the cellulose raw material produces high tenacity rayon by using ionic liquids, for example, 1-butyl-3-methylimidazolium chloride or other solvents such as ammonia or ammonium thiocyanate, as dissolving or suspending agents. In still another modified process, a blowing agent or air is added to produce “hollow” rayon fibers. As described above, a number of conventional synthetic routes are available to produce rayon fibers.

Even in the standard, viscose process for making regular rayon, process changes and/or additives can be introduced to synthesize rayon having properties that would be preferred for tampon performance. For example, certain nitrogen and oxygen based modifiers are added to modify an amount of orienting stretch imparted to the fiber. Additionally, dimethylamine (DMA) can be introduced to form dimethyldithiocacarbamate, an effective agent in modifying viscose. In one embodiment, DMA is added to the salt-acid spin bath (at Step 210 of FIG. 1) to produce an appropriate level of zinc crosslinking.

The inventors have recognized that of these synthetic routes, the viscose rayon forming process, described above with reference to FIG. 1, provides preferred results due, in part, to practical economic and manufacturing considerations. However, the inventors also recognize that the use of NMMO and ionic liquids as solvents provide preferred environmental results, since the synthetic routes typically employ solvent recycling. Moreover, synthetic routes using NMMO and ionic liquids are becoming increasingly more economical and provide means for crosslinking and tailoring rayon microstructures (e.g., molecular weight and degree of crystallinity) that viscose synthetic routes do not easily permit. Accordingly, the inventors have recognized that differing synthetic routes may be employed to achieve needs of differing tampon applications.

The inventors have also discovered that varying specific synthetic details (e.g., time, temperature, humidity, pressure settings, and the like) within the above-described synthetic routes improves product performance and particularly when, as the inventors have discovered, eucalyptus pulp is employed as the cellulose raw material. For example, the inventors have discovered that the amount of time cellulosic raw material pulp sheets are steeped in caustic soda, dried, shredded, and pre-aged, as well as the temperature and humidity settings, affects the amount of oxidative degradation and thus, affects overall rayon average molecular weight. Moreover, the inventors have discovered that methods used to extrude, stretch and crimp filaments, and the size and shape of spinnerets affect the morphology, orientation and degree of crystallinity of the rayon being produced. The inventors have also discovered that producing rayon using viscose processes and employing Y-shaped spinnerets provides high absorbency.

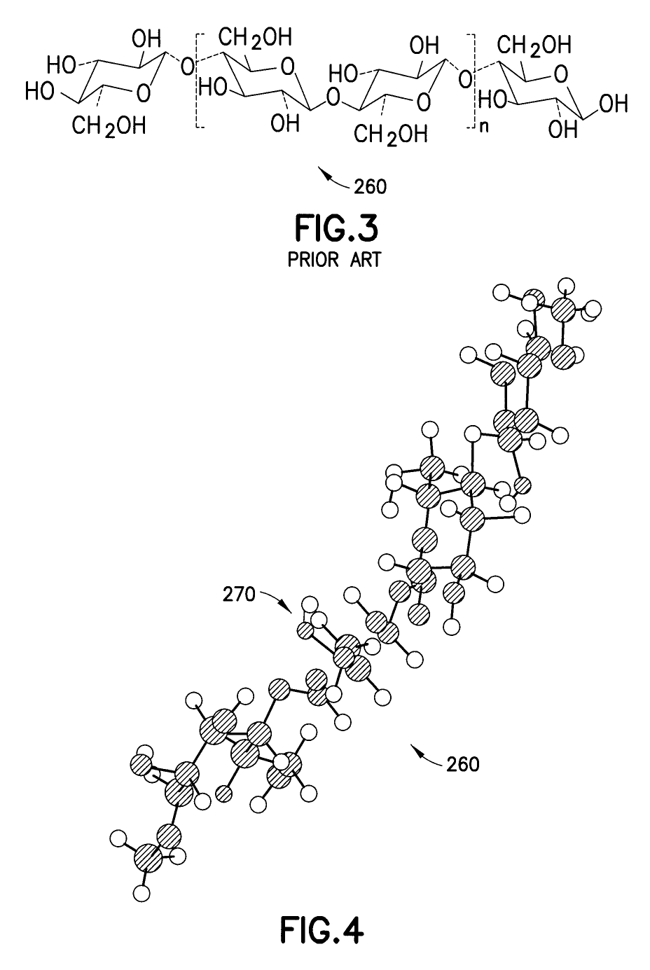

FIGS. 3-6 illustrate certain aspects of cellulosic chemistry as well as typical properties of rayon made by conventional means that are evaluated and refined by, for example, modifying the process steps illustrated in FIG. 1, to provide a superior grade of rayon adapted to requirements of tampon products. FIGS. 3 and 4 illustrate the known chemistry of cellulose. As shown in FIGS. 3 and 4, cellulose 260 is comprised of repeating units of D-glucose, which are six-membered rings known as “pyranoses.” The pyranose rings are joined by single oxygen atoms (acetal linkages) between one of the carbons of one of the pyranose rings and a different carbon on an adjacent pyranose ring. Since a molecule of water is lost when an alcohol and a hemiacetal react to form an acetal, the glucose units in the cellulose molecule are referred to as “anhydroglucose” units. As shown in FIG. 3, the internal anhydroglucose units each have three (3) alcoholic groups (e.g., —OH groups), while end anhydroglucose units of the long chain molecule have four (4) alcoholic groups.

One aspect of the acetal linkage that is important is the spatial arrangement. When glucose forms a first pyranose ring, the hydroxyl group on one carbon of the first ring can approach the carbonyl on a second ring from either side and thus, result in different stereochemistries. For example, in one stereochemistry with functional groups in equatorial positions, the molecular chain of cellulose extends in a straight line making it a good fiber-forming polymer. In a slightly alternative chemistry with the linkage in an axial position, starch molecules are formed which tend to coil rather than extend.

With so many —OH groups in a molecule, one would expect that cellulose is water-soluble. But it is not. Because of the equatorial positions of these hydroxyls on the cellulose chain, they protrude laterally along the extended molecule as shown generally at 270 of FIG. 4. This positioning makes them readily available for hydrogen bonding. These strong hydrogen bonds produced several key properties of cellulose, namely: 1) the bonds prevent penetration of the solid cellulose by aqueous solvents, resulting in a lack of solubility not only in water, but in almost all other solvents; 2) the bonds cause the chains to group together in highly ordered structures (e.g., crystal like structures); 3) the bonds provide high strength; and 4) the hydrogen bonds also prevent cellulose from melting, like most thermoplastics ordinarily do.

But cellulose is not entirely crystalline. Typically, the cellulose chains are usually longer than the crystalline regions. Thus, there are regions of both order (i.e. crystalline regions) and disorder (i.e. amorphous regions). In less ordered regions, the chains are further apart and more available for hydrogen bonding to other molecules, such as water. Most cellulosic structures, rayon included, can absorb large amounts of water. Thus, rayon does not dissolve in water, but it does swell in it readily.

In view thereof, the inventors have recognized that a key to synthesizing a good grade of rayon for tampon performance requires a proper “balancing” of the cellulose structure. For example, the rayon must have enough disorder to get good absorbency and wicking of aqueous-based fluids such as menses, while retaining enough crystalline structure to maintain good strength especially once the rayon has been wetted and to allow the fibers to be formed stably in a viable, economic, manufacturing process. The inventors have recognized that a number of synthesis guidelines can be followed to achieve the aforementioned balancing.

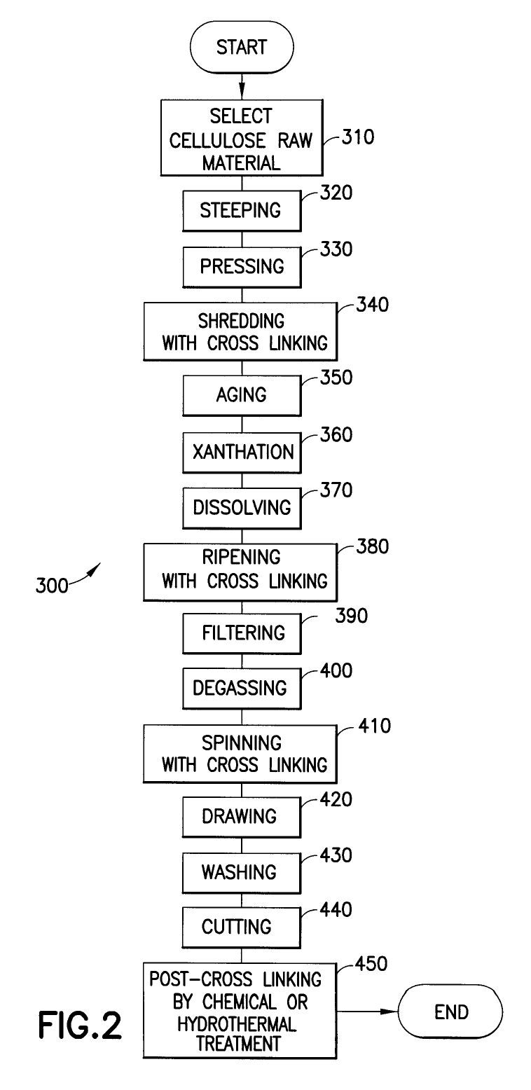

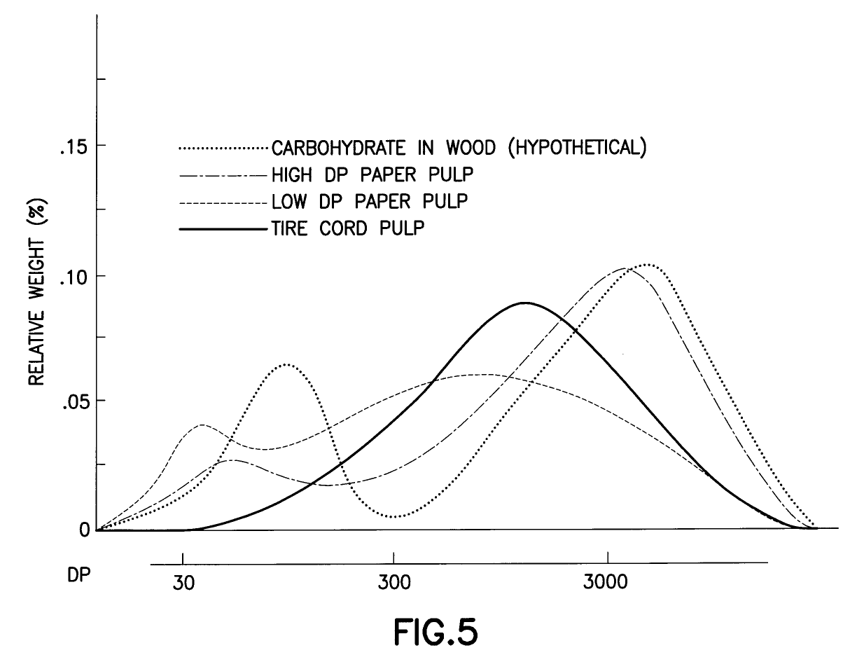

As described above, in order for fibers to be formed the molecular weight of standard cellulose is first lowered from that of pulp (FIG. 5) to a level such that extrusion through relatively small spinerettes is technically possible and economically feasible. As FIG. 5 illustrates, typical pulp degrees of polymerization (DP) range from about 30 to over 3000. By comparison, the degree of polymerization of rayon is only about 260. As noted above with respect to the conventional process 100 of manufacturing viscose rayon (FIG. 1) and as described below with respect to an improved manufacturing process 300 of FIG. 2, several steps accomplish this lowering of molecular weight. First, a suitable choice of a raw material is made (at Blocks 110, 310). Second, as the pulp is “steeped (at Blocks 120, 320) in caustic and then pressed (at Blocks 130, 330), there is some oxidative degradation and alkaline hydrolysis to reduce the molecular weight to an acceptable level for processing.

The degree of crystallinity can be controlled in several steps in the manufacture of rayon. There are three (3) hydroxyl groups available on each internal anhydroglucose ring but, given the discussion above, the inventors have recognized that it is difficult to react all (3n+2) of these groups, where n is the degree of cellulosic polymerization. For example, the hydrogen bonding is so strong that reactions to disrupt that bonding tend to be sterically limited. Thus, in the xanthation step (Block 160, 360), the degree of substitution (DS) is typically only about seven tenths (0.7), for example, about seventy percent (70%) of the hydroxyls are typically reacted. Many of the hydroxyls that are relatively easy to react are in the less ordered regions. Higher degrees of xanthate substitution can disrupt the crystalline regions. The inventors have noted that this can interfere with the inter-chain hydrogen bonds and, in a subsequent step, lower the fiber wet tenacity and strength.

The inventors have discovered that one way to change cellulosic microstructure is to, for example, add a relatively small amount of crosslinking agent (about one tenth of one percent (0.1%) or less) just after the xanthation reaction (Blocks 160, 360), in order to provide some intermolecular and intramolecular crosslinks involving unsubstituted —OH groups. Crosslinking levels should be low at this stage so as to allow subsequent steps of dissolving (at Blocks 170, 370), ripening (at Blocks 180, 380) and filtration (at Blocks 190, 390) to occur.

The inventors have recognized that another step where crosslinking agents may be added is a spinning step (e.g., Blocks 210, 410). For example, one conventional process developed by Courtaulds North America, Inc. (Mobile, Ala., USA) (“Courtaulds”) used small amounts of formaldehyde in the spin bath to develop a fiber called W-63 that had unusually high tenacity and modulus (e.g., about 7-10 g/den). Based on this technology Courtaulds produced a yarn called “Tenex.” However, there are perceived deficiencies with the Tenex yarn. For example, the fiber was too brittle and there were problems associated with recovery of the fiber from the spin bath. Thus, the inventors have recognized that to achieve the balance act of crystallinity, water absorption, wet strength and fiber formability, special spinning conditions and spin modifiers such as those outlined above could be added to the manufacturing process (at Blocks 210, 410) to affect the degree of crystallinity. Also, during the drawing step (at Blocks 220, 420), the rate of drawing can be changed in order to change the crystallinity of the filaments. The degree of stretch imparts some orientation, hence influences the degree of crystallinity, to the fibers made at this stage.

Additionally, post crosslinking agents could be added to fibers, for example, after the fibers have been drawn (at Blocks 220, 420) or before a final washing step (at Blocks 230, 430). The inventor notes that crosslinking at these later stages (e.g., at Blocks 420 or 430) can help produce a stronger, tougher fiber and hence a stronger, tougher web used in tampon manufacture.

The inventors have also discovered that the choice of crosslinking agents is a significant factor in the formation of improved rayon materials. For example, conventional processes typically employ formaldehyde as a crosslinking agent preferring cost and efficiency considerations. Moreover, the inventor notes that there is a perceived disadvantage from a safety prospective with the use of formaldehyde in a product that will be used in a human body. Accordingly, the inventors favor use of citric acids as cellulosic crosslinking agents. The inventors have found that to crosslink cellulose effectively, at least two hydroxyl groups should be combined in a cellulose molecule (e.g., intramolecular crosslinking) or in adjacent cellulose molecules (e.g., intermolecular crosslinking). Effective crosslinking typically requires that the crosslinking agent be difunctional (e.g., 1,3-Dichloro-2-propanol) with respect to cellulose for reaction with the two hydroxyl groups. As an alternative to a single difunctional crosslinking agent, a mix of two or more different molecules can be employed to provide an effective difunctional and multifunctional crosslinking. For example, in one embodiment, a crosslinking agent may include glyoxal as well as a glyoxal-derived resin. In one embodiment, a cyclic urea/glyoxal/polyol condensate (e.g., sold under the designation SUNREZ 700M by Sequa Chemicals, Inc., Chester, S.C. USA) provides a multifunctional crosslinking agent.

Other examples of crosslinking agents are familiar to those skilled in the art. Since zinc salts are typically used in the spin bath (at Blocks 210, 410), ionic crosslinkers involving zinc sulfates and similar divalent cations and appropriate anions may be used. Other crosslinking agents would include, but are not be limited to, butanetetracarboxylic acid, cyclobutane tetracarboxylic acid, tetramethylenebisethylene urea, tetramethylenedidisocyanate urea, polymeric polyacids such as polymethacrylic acid, methylated derivatives of urea or melanine such as dimethyloldihydroxyethyleneurea, glutaraldehyde, ethylene glycol bis-(anhydrotrimellitate) resin compositions, and hydrated ethylene glycol bis-(anhydrotrimellitate) resin compositions.

The inventors have recognized that the choice of a particular crosslinking agent for tampon applications depends on a variety of factors. Besides achieving the crystallinity/wet strength/absorbency/fiber formability “balance” discussed herein, the choice of chemistry used depends upon such other factors as, for example: product health and safety, regulatory approvals, product quality; sufficiently high reaction rates at temperatures of interest, the propensity of undesirable side reactions, manufacturing issues, raw material cost of particular crosslinking agent, and the like.

The inventors have recognized that crosslinking is likely to take place, to a greater extent, in crystalline fractions of the cellulose rather than in the non-crystalline fractions. This result is apparently seen because polymer segments are closer together in crystallites since the chain packing density is greater. Thus, interaction of crystallinity and crosslinking is expected. The inventors have recognized that such an interaction influences key polymer properties, such as tampon performance.

The inventors have also discovered that in addition to the choice of a crosslinking agent, the amount of crosslinking agent used is relevant. For example, the inventors have discovered that the amount of a crosslinking agent that is used may be dependent upon the degree of crosslinking desired, the efficiency of the crosslinking reaction and the desired molecular weight between crosslinks that would produce enhanced wet bulk and enhanced tampon properties that would accrue from the reaction. The inventors have found that a level of crosslinking agent used ranges from a value of about one thousandth of one percent (0.001%) to a value of about twenty percent (20%), based on a total amount of cellulose present to be treated. In one embodiment, a crosslinking agent would be present in an amount of about five percent (5%) by weight based on the total weight of cellulose fibers. With respect to the efficiency of the cross linking reaction, the inventors have determined that, like most chemical reactions, there is a temperature that is most optimal for the particular chemical reaction of interest. In many cases the crosslinking reaction proceeds reasonably rapidly at the same temperature at which rayon is normally processed in the steps outlined with reference to the convention process 100 of FIG. 1. In other cases, it is desirable to add a catalyst to promote the reaction either by free-radical means or by an oxidation-reduction catalytic reaction. General examples of catalysts include, for example, peroxides, perchlorates, persulfates, and/or hypophosphites.

In another aspect of the present invention, the inventor selectively introduces the crosslinking reaction to the rayon synthesis process. An improved viscous rayon forming process 300 is illustrated in FIG. 2, and is similar to the aforementioned viscous rayon forming process 100 of FIG. 1, where like steps of the improved forming process 300 having reference numerals prefixed by “3” and “4” correspond to steps prefixed “1” and “2”, respectively, of the conventional rayon forming process 100 of FIG. 1. As shown in FIG. 2, the crosslinking reaction may be introduced early in, for example, the viscose “ripening” reaction (e.g., at Block 380 of FIG. 2) or during the introduction of a solvent or slurry agent (e.g., NMMO) to the shredded pulp pieces (e.g., at Block 340 of FIG. 2). Alternatively, crosslinking can be carried out later in the viscous reaction such as, for example, after the degraded rayon cellulose has been largely formed (e.g., at Block 410 of FIG. 2). Crosslinking reactions can also be employed on the developing, coagulating fiber filaments, the finished fiber tow, cut rayon fibers or on carded webs produced from the finished rayon fibers.

Additionally, it is within the scope of the present invention to employ wet and dry crosslinking reactions. Dry crosslinking may be performed when the cellulose is in a collapsed state where it is substantially free of water and moisture (e.g., within the pressing step at Block 330 of FIG. 2). Wet crosslinking may be performed with the cellulose in a swollen or wet state. In one embodiment, the crosslinking process is performed on finished but swollen staple fibers (e.g., after cutting at Block 440 of FIG. 2), prior to web formation. In this manner unused crosslinking agents could be dispersed in a suitable solvent, treated at high temperature in an oven or like vessel at, for example, about one hundred degrees Celsius (100° C.) for about one (1) hour, to complete the crosslinking reaction and optimally increase the wet bulk properties. The crosslinking agents, crosslinking catalysts (if any), and polar solvents are washed out with water and thoroughly dried prior to web formation and tampon forming.

It is also within the scope of the present invention to vary the amount and type of crosslinking catalysts used to speed up the crosslinking reactions. In addition to those listed above, the inventors have discovered that preferred cellulose crosslinking catalysts include, for example: magnesium chloride or magnesium nitrate; zinc chloride, zinc nitrate, or zinc fluoroborate; lactic acid, tartaric acid or hydrochloric acid; ammonium sulfate or ammonium phosphate; or amine hydrochlorides. In one embodiment, crosslinking catalyst levels range from about a thousandth of one percent (0.001%) to about ten percent (10%) by weight based on a total weight of cellulose fibers to be treated. It should be appreciated, however, that it is not a necessary step in the crosslinking reaction to introduce a crosslinking catalyst. Accordingly, it is within the scope of the present invention to conduct crosslinking reactions without the use of a crosslinking catalyst.

The inventors have discovered that one or more of the ingredients used above as part of the crosslinking reaction impart secondary advantages when employed within tampons products. For example, ingredients such as glycerol monolaurate, sorbitan monolaurate (Tween 20), sodium lauryl sulfate, sodium dioctyl sulfosuccinate, potassium oleate, and other surfactants, provide an anti-bacterial action. These ingredients may also be beneficial in assisting fiber finishing as the ingredients have surface-active properties that affect fiber surface properties, interaction and thus absorption of menses. Moreover, surfactants such as these ingredients could be used to improve the wettability of cellulose and thus promote the substitution and crosslinking reactions as well. Finally, these same ingredients promote as fiber-fiber friction and cohesion force that, in turn, contribute to effective processing of fibers into webs.

As shown in FIG. 2, at Block 450, it is within the scope of the present invention to employ post-crosslinking by chemical or hydrothermal treatment to further improve the strength of the fiber. Post-crosslinking is described further below.

It should be appreciated that the above described improvements to the rayon synthesis process provide a number of factors or “levers” that can be tuned and adjusted by product developers to achieve a desired “balance” of rayon properties for particular tampon applications. As noted above, to maximize performance different types of tampons require different rayon properties. For example, tampons rated “light” and/or “regular” absorbency include rayon having less absorbency, less crosslink density, and greater crystallinity. Accordingly, the inventors have found that by expanding the duration of the drawing step conducted at Block 420 of FIG. 2, cellulose chains are lengthened and interchain hydrogen bonds are formed to provide greater areas of crystallinity within the rayon fiber and thus provide rayon tailored more toward light and regular absorbency applications. Tampons rated “super” and/or “super plus” absorbency include rayon having a relatively higher gram per gram syngyna absorbency, relatively higher crosslink density and a greater amorphous polymer fraction.

As illustrated above, in one aspect of the invention the inventors have discovered that by adjusting the various factors described above, interactions within the rayon synthesis process may be controlled and optimized to provide improved synthesis processes and, as a result, improved rayon for use in tampon pledgets. The inventors have determined that the optimized synthesis processes result in rayon having a number of desirable properties. For example, the inventors have discovered that by adjusting one or more of the aforementioned factors the synthesis process may be tailored to improve tampon absorbency capacity and wicking rate, improve fiber physical properties (e.g., polymeric microstructure including the degree of crystallinity, molecular weight distributions, and reduce levels of unreacted impurities and byproducts), and fiber surface properties.

In one embodiment, conventional test analyses and methods may be employed in a novel manner to determine, as described herein, key attributes of the inventive process 300 of making modified rayon. For example, to determine the crystallinity of the treated samples at different conditions, a sample is placed into a chamber of an analytical x-ray diffractometer and scanned using an appropriate level of x-ray energy and intensity for a sufficient length of time to get a signal. X-ray diffraction photographs of cellulose show both a regular pattern, characteristic of the crystalline portion, and a diffuse halo, characteristic of the amorphous material. Besides the x-ray methods, density methods, NMR, infrared absorption and other methods can be used to infer the degree of crystallinity.

Similarly, absorbency can be determined in accordance with prior art methods. There are standard methods for determining absorbency, for example, INDA Test Method IST 10.1 (5), “Standard Test Method for Absorbency Time, Absorbency Capacity, and Wicking Rate,” Association of the Nonwoven Fabrics Industry, Cary, N.C., 1995. For tampons, there is also the FDA-mandated Syngyna test method (Federal Register, Volume 54, Number 206, pp. 43773-43774).

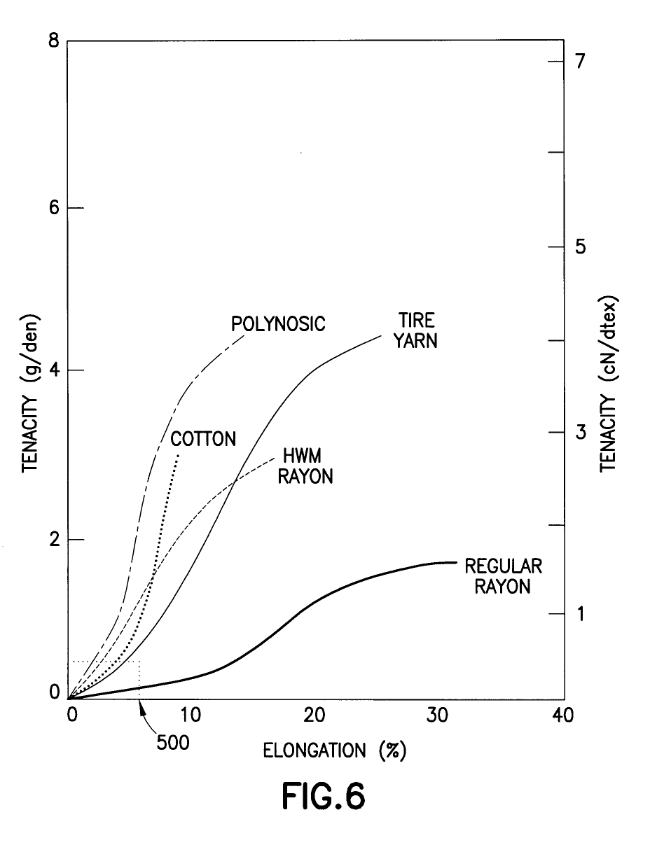

Moreover, for fiber tenacity (dry or wet strength), there are a variety of test methods. For example, ASTM D 2256-95a, “Standard Test Method for Tensile Properties of Yarns by the Single Strand Method,” is one such standard test methodology. This and similar test methods could be performed using instruments available at, for example, Instron (825 University Ave, Norwood, Mass., U.S.A.; www.instron.com). FIG. 6 shows results as a plot of tenacity versus percent elongation for various rayon grades. Fibers of the present invention exhibit wet strengths that are typically higher than regular rayon but not as high as the some other grades, for example, wet tenacity at five percent (5%) elongation would be about five tenths of one gram (0.5) per denier for rayon of the present invention, as illustrated generally at 500 of FIG. 6.

Dynamic mechanical analysis methods are useful to evaluating mechanical properties of crosslinked polymers that may exhibit both elastic (solid-like) and inelastic (liquid-like) properties. Such viscoelastic methods are typically used to evaluate the extent to which a polymer has been crosslinked.

Further, gel permeation chromatography (GPC), solution viscosity, high pressure liquid chromatography (HPLC), and other standard analytical methods such as gas chromatography, simple titrations and solubility determinations) can be used to analyze the molecular characteristics of the present invention. The first two analytical methods are useful for determining the cellulose molecular weight; whereas the latter methods are used to determine the concentration of unreacted small molecular species that may present themselves during the various crosslinking reactions described herein.

The inventors analyzed a number of exemplary fibers to illustrate various features of the present invention. In the examples provided below treatments were applied to a viscose rayon fiber such as, for example, a Kelheim Multilobal fiber sold under the brand name GALAXY by Kelheim Fibres, Ltd., Kelheim, Germany. Chemical and/or hydrothermal treatments were applied to the viscose rayon fiber.

High Temperature Wet Treatment of Viscose Rayon Fibers

Procedures for High Temperature Wet Treatment (hydrothermal treatment)

Pre-treatment—The viscose rayon fiber is first washed three (3) times with distilled water at a room temperature of about twenty-three degrees Celsius (23° C.) to remove any lubricating agents (fiber finish). The fiber is then dried by compressing and placing in a vacuum oven at about sixty degrees Celsius (60° C.) overnight.

High temperature wet treatment (HTWT)—In an embodiment, a temperature range of about ninety to about one hundred fifty degrees Celsius (90 to 150° C.) is used. In another embodiment, a temperature range of about one hundred to about one hundred twenty-four degrees Celsius (100 to 124° C.) is used for the high temperature wet treatment. Each includes the following steps.

1. In an autoclave, an about one thousand milliliter (1000 ml) water bath was preheated to a temperature of about one hundred degrees Celsius (100° C.).

2. Twenty grams (20 g) of the viscose rayon fiber was immersed in the water bath. The autoclave was then immediately sealed. The water bath temperature was monitored. When the temperature reached a target temperature, a stopwatch was started.

3. The fiber sample is keep at a setting temperature level for a desired time period.

4. Then, the pressure of autoclave is released, and the fiber sample was removed and then soaked in a one thousand milliliter (1000 ml) distilled water bath at about twenty-three degrees Celsius (23° C.) for about five (5) minutes.

5. After that, the fiber sample is dried by compressing and placing the sample in a vacuum oven at a temperature of about sixty degrees Celsius (60° C.) overnight.

Note: Some time was taken to heat up to the desired target temperature. The time value ranged from about fifteen to about forty (15-40) minutes to heat up to the target temperatures, which ranged in the examples provided below from about one hundred and eight degrees Celsius to about one hundred twenty-four degrees Celsius (108° C.-124° C.).

The above described procedures were repeated until a desired amount of fiber sample was prepared for evaluation. In one embodiment, the desired amount of fiber sample was about one hundred (100) grams.

Procedures for Chemically Crosslinking Treatment (CCT)

Pre-treatment

Rayon viscose fiber was first washed three times with distilled water at a room temperature of about twenty-three degrees Celsius (23° C.) to remove the fiber finish, i.e. lubricating agent. It was then dried by compressing and placing in a vacuum oven at a temperature of about sixty degrees Celsius (60° C.) overnight. The pre-treated rayon fiber was used for a sample preparation.

Chemically crosslinking treatments

Six different crosslinking chemical agent systems were investigated for the chemically crosslinking treatment (CCT) of viscose rayon fibers. The CCT procedures using each crosslinking agent system, are described below.

Polycarboxylic acids

Polycarboxylic acids such as, for example, 1,2,3,4-Butanetetracarboxylic acid and citric acid are used as crosslinkers through esterification reactions with the hydroxyl groups of cellulose in the presence of catalysts.

A. 1,2,3,4-Butanetetracarboxylic acid

Crosslinking system

Crosslinking agent: 1,2,3,4-butanetetracarboxylic acid (BTCA),

Catalyst: sodium hypophosphite monohydrate NaH2PO2.H20

B. Citric Acid

Crosslinking system

Crosslinking agent: citric acid (CA)

Catalyst: sodium hypophosphite monohydrate NaH2PO2.H20

Procedures for Small Trials

1. At room temperature, eleven grams (11 g) of rayon fiber was immersed in an about two hundred twenty milliliters (220 ml) of an aqueous solution containing 1,2,3,4-Butanetetracarboxylic acid or citric acid (about one to five percent by weight (1 to 5 wt %) based on the weight of rayon fiber) and about one to five percent by weight (1 to 5 wt %) of sodium hypophosphite for about ten minutes (10 min.).

2. After about ten minutes (10 min.), the fiber was pressed to remove most of liquid and then dried at about fifty to sixty degrees Celsius (50-60° C.) in a vacuum oven, to a level containing a desired amount of liquid, e.g., about twenty-five percent by weight (25 wt %) or about fifty percent by weight (50 wt %) based on the dry fiber basis.

3. Then the fiber was cured at about one hundred sixty-five to about one hundred seventy degrees Celsius (165 to 170° C.) for about two minutes (2 min.).

4. The cured fiber was washed three (3) times with distilled water to remove the unreacted acid and catalyst. At each wash, the cured fiber was washed for about five minutes (5 min) in about two hundred twenty milliliters (220 ml) of distilled water. Once washed, the fiber is then fully dried in a vacuum oven at a temperature of about sixty degrees Celsius (60° C.).

Dimethyldihydroxyethylene urea

Crosslinking system

Crosslinking agent: modified formaldehyde-free agent—dimethyldihydroxyethylene urea (DMDHEU).

Catalyst: MgCl2

Procedures for Small Trials

1. At room temperature, eleven grams (11 g) of rayon fiber was immersed in an about two hundred twenty milliliters (220 ml) aqueous solution containing DMDHEU (one or five percent by weight (1 or 5 wt %) based on the weight of rayon fiber) and one to five percent by weight (1-5 wt %) of MgCl2 for about ten minutes (10 min.).

2. After about ten minutes (10 min.), the fiber was pressed to remove most of liquid and then dried in a vacuum oven at a temperature of between about fifty to sixty degrees Celsius (50-60° C.), to a level containing a desired amount of liquid, e.g., about twenty-five or fifty percent by weight (25 or 50 wt %) based on the dry fiber basis.

3. Then the fiber was cured at about one hundred sixty-five to about one hundred seventy degrees Celsius (165 to 170° C.) for about two minutes (2 min).

4. The cured fiber was washed three (3) times with distilled water to remove the unreacted crosslinking agent and catalyst. At each wash, the cured fiber was washed for about five minutes (5 min) in about two hundred twenty milliliters (220 ml) of distilled water. Once washed, the fiber is then fully dried in a vacuum oven at a temperature of about sixty degrees Celsius (60° C.).

2,4-dichloro-6-hydroxy-1,3,5-triazine

Crosslinking system

Crosslinking agent: 2,4-dichloro-6-hydroxy-1,3,5-triazine (DCH-Triazine)

Catalyst: NaHCO3 (for pH adjustment)

As an initial step, a water-soluble DCH-Triazine sodium salt was prepared by reacting cyanuric chloride with NaOH at a low temperature.

Procedures for Small Trials

At room temperature, about eleven grams (11 g) of rayon fiber was immersed in an about two hundred twenty milliliters (220 ml) aqueous solution containing DCH-Triazine sodium salt (one to five percent by weight (1 to 5 wt %) based on the weight of rayon fiber) and one to five percent by weight (1 to 5 wt %) of NaHCO3 for about ten minutes (10 min).

After about ten minutes (10 min), the fiber was pressed to remove most of liquid and then dried in a vacuum oven at a temperature of between about fifty to sixty degrees Celsius (50-60° C.), to a level containing desired amount of liquid, e.g., about twenty-five or fifty percent by weight (25 or 50 wt %) based on the dry fiber basis.

Then the fiber was cured at about one hundred sixty-five to about one hundred fifty to about one hundred sixty degrees Celsius (150 to 160° C.) for about two minutes (2 min).

The cured fiber was neutralized with about two hundred twenty milliliters (220 ml) of two percent by weight (2 wt %) of acetic acid.

The cured fiber was washed three (3) times with distilled water to remove the unreacted crosslinking agent and catalyst. At each wash, the cured fiber was washed for about five minutes (5 min) in about two hundred twenty milliliters (220 ml) of distilled water. Once washed, the fiber is then fully dried in a vacuum oven at a temperature of about sixty degrees Celsius (60° C.).

Glyoxal/Glyoxal Derivative Resin

Crosslinking system

Crosslinking agent: glyoxal and glyoxal derivative resin

Catalyst: MgCl2

Glyoxal resin preparation

A cyclic urea/glyoxal/polyol condensate (Glyoxal resin) is prepared by reacting glyoxal, cyclic urea and polyol. The detailed procedure is as the following.

To an about one liter flask sixty (60) parts (1.0 mole) urea, seventy-five (75) parts of water, seventy-five (75) parts of 1,4-dioxane, sixty (60) parts (1.0 mole) of aqueous formaldehyde, and seventy-two (72) parts (1.0 mole) of isobutyraldehyde were added. The reaction mixture was stirred and heated at about fifty degrees Celsius (50° C.) for about two (2) hours.

Following the addition of a catalytic amount of acid, the reaction mixture is heated at its reflux temperature for about six (6) hours. The product is a clear solution that contained 4-hydroxy-5,5-dimethyltetrahydropyrimidin-2-one. The inventors confirmed this by IR spectroscopy, identifying peaks at 3300 cm−1 as NH or OH moeties, 1660 cm−1 as C═O, and 1075 cm−1 as C—O.

The above product was heated with one hundred fifty (150) parts (1.08 moles) of forty percent (40%) glyoxal and thirty-two (32) parts (0.4 mole) of propylene glycol at a temperature of about seventy degrees Celsius (70° C.) for about four (4) hours to form the cyclic urea/glyoxal/polyol condensate (Glyoxal resin).

Procedures for Small Trials

1. At room temperature, about eleven grams (11 g) of rayon fiber was immersed in an about two hundred twenty milliliters (220 ml) of an aqueous solution containing glyoxal (one to five percent by weight (1 to 5 wt %) based on the weight of rayon fiber), glyoxal resin (one to five percent by weight (1 to 5 wt %) based on the weight of rayon fiber), and one to five percent by weight (1 to 5 wt %) of MgCl2, for about ten minutes (10 min).

2. After about ten minutes (10 min), the fiber was pressed to remove most of liquid and then dried in a vacuum oven at a temperature of between about fifty to sixty degrees Celsius (50-60° C.), to a level containing desired amount of liquid, e.g., about twenty-five or fifty percent by weight (25 or 50 wt %) based on the dry fiber basis.

3. Then the fiber was cured at about one hundred sixty degrees Celsius (160° C.) for about two minutes (2 min).

4. The cured fiber was washed three (3) times with distilled water to remove the unreacted crosslinking agent and catalyst. At each wash, the cured fiber was washed for about five minutes (5 min) in about two hundred twenty milliliters (220 ml) of distilled water. Once washed, the fiber is then fully dried in a vacuum oven at a temperature of about sixty degrees Celsius (60° C.).

Ethylene glycol-diglycidylether (EDGE)

Crosslinking system

Crosslinking agent: ethylene glycol-diglycidylether (EDGE)

Catalyst: NaOH

Procedures for Small Trials

1. About eleven grams (11 g) of rayon fiber was immersed in an about two hundred twenty milliliters (220 ml) of an aqueous solution containing EDGE (one to seven percent by weight (1 to 7 wt %) based on the weight of rayon fiber), and one to two percent by weight (1 to 2 wt %) of NaOH, for about four to six hours (4-6 hrs) at about forty degrees Celsius (40° C.).

2. The treated fiber is washed three (3) times with distilled water to remove the unreacted crosslinking agent and catalyst. At each wash, the cured fiber was washed for about five minutes (5 min) in about two hundred twenty milliliters (220 ml) of distilled water. Once washed, the fiber is then fully dried in a vacuum oven at a temperature of about sixty degrees Celsius (60° C.).

In all the CCT preparations described above, procedures were repeated in order to obtain enough treated fiber for evaluations, usually this was about one hundred (100) grams.

Procedures for Evaluation of Crosslinked Rayon Fibers

Multilobal fibers (Kelheim fibers) that have been chemically or hydrothermally crosslinked by a variety of treatments were usually checked versus appropriate controls (usually untreated Kelheim Galaxy fiber). The inventors evaluated the fibers using the “bagged pledget” test method, using special nonwoven bags. Procedures for making up these nonwoven bags are described below.

For each example, typically about twenty-five (25) bagged tampons were made by the methods described below for each “cell”, for example, each aliquot of hydrothermally or chemically crosslinked rayon or a control sample of fiber.

Procedures for Making Bagged Tampons

1. Obtain a sufficient number of bags to enclose the loose rayon fiber.

2. Obtain a sufficient number of commercial tampon such as, for example, GENTLE GLIDE super white applicators (barrels and plungers) as well as a sufficient supply of standard string (gentle glide is a registered trademark of Playtex Products, Inc., Shelton, Conn., USA). Also, collect together the fiber samples to be tested.

3. Collect a supply of standard multilobal rayon as control samples.

4. From the bags and fibers above, typically several “cells” would be run at a time, each with about twenty-five plus (25+) tampons. Operators were instructed to handle the fiber using rubber gloves.

For each of the cells:

5. At least twenty-five plus (25+) aliquots of 2.7+/−0.1 grams of the selected (absorbent) fiber variant were weighed out into containers such as, for example, aluminum muffin tins. In one series, for example, there were twenty-five plus (25+) aliquots (“fluffballs”) weighed out for eight (8) different cells to provide about two hundred (200) weigh-ups in all.

6. For each of these aliquots a Hauni HP simulator was set up for forming super tampons. Standard operations for forming using this simulator from nonwoven webs are provided below. These instructions provide one example of machine settings and the general sequence of operation. Steps 7-19 below are used specifically for forming bagged tampons from fibers.

7. Using the preweighed out fluffballs, form the fluffball by pushing small amounts of the fluffball into the transfer throat of the HP Simulator until the entire fluffball is in the transfer throat, which is about 0.527 inch in diameter.

8. The fluffball was then transferred into a hot oven tube, preheated at about two hundred sixty degrees Fahrenheit (260° F., 127° C.). The oven tube diameter was about 0.495 inch.

9. The hot oven tube was compressed on a Domer, as is generally known in the art. Then the pledget was re-positioned. The heated “Dome” fixture was turned around so that the flat shaft-like back end of the fixture actually presses against the pledget in the oven tube. The flat pusher end of the air cylinder has two spacers on it: one is about one half inch (0.5 in.) and the other is about three sixteenth inch (0.187 in).

10. The warmed pledget in the oven tube is then placed into a conveying oven at about five hundred twenty-five degrees Fahrenheit (525° F., 274° C.), with a speed of about thirty-six and one half (36.5) inches per minute. The conveying oven is generally known in the art.

11. The hot oven tube is then taken back to the Hauni HP Simulator.

12. Put the right nonwoven bag having a length of about two to about two and one quarter inches (2-2.25 in.) long, inside out, over the end of an “upside down” cold oven tube (0.531″ in diameter). This second, cold oven tube is “cold” because it has not been preheated. The cold oven tube is placed onto a transfer station on the HP Simulator.

13. Remove the pledget from the hot oven tube and put the oven tube back into the warm oven, which is maintained at a temperature of about two hundred sixty degrees Fahrenheit (260° F., 127° C.).

14. The hot formed pledget is then placed into the transfer throat. It is then transferred into the cold oven tube through the bag. This will push the bag and the pledget into the cold oven tube.

15. Transfer the bagged pledget from the cold oven tube into the stringer chain with the open end of the bag at the “stringing” end of the chain link.

16. The string is then put through the bottom of the pledget.

17. The excess open portion of the bag is then folded into the middle.

18. The flat bag end is folded down to the end of the pledget. Then a knot is tied to secure the string to the pledget.

19. The formed, strung pledget is then transferred, using air cylinder pressure, to a super GENTLE GLIDE white applicator.

20. Steps 5-19 are repeated a sufficient number of times to make the twenty-five plus (25+) tampons for the cell of interest. Then the tampons are placed into a large polyethylene bag for each cell. Each bag is then labeled with the particular cell number, including a short description of which fiber treatment was used, if any, for the particular cell.

Two tests were done to demonstrate aspects of the present invention, the standard Syngyna testing for absorbency and moisture testing. The procedure for Syngyna testing is provided below. Moisture testing, e.g., a loss of weight on drying, was done using a Mettler-Toledo Halogen Analyzer, Model No. MR-73. Three to five replicate moisture analyses were typically done for each example.

Preparation of Bags Used in the Bagged Pledget Forming Tests Described Above

The following descriptions outline exemplary methods for preparing nonwoven bags used to evaluate small amounts of different fibers. Four different types of nonwoven material were used to make bags in experiments described herein. Although, the inventors did not observe any differences in results obtained that would be attributed to different types of bags used.

The nonwoven material used for many of the examples described herein was a “cover stock” type of nonwoven material designated in the tables below as “PGI-1,” which is a 0.5 oz. per sq. yd. material sold as BiCo #4139 by PGI (Chicopee, Ark.). A variant of the PGI nonwoven web, prepared at a slightly lower basis weight, was used and is labeled in the tables below as “PGI-2,” which is a 0.4 oz. per square yard material. Also, some nonwoven bags, labeled as “BDK,” were made from material purchased from BDK Nonwovens (NC, USA) under Style number 1014, R-73763. Finally, some bags were made using a spunbond polyethylene/polyester heat-sealable nonwoven blend, labeled “HDK” in the tables below, 16 gsm, available from HDK Industries, Inc. (Rogersville, Tenn. USA).

Cutting:

1. Coverstock should be cut to the right size. A sample of an appropriate coverstock nonwoven (one of the three described above) should be cut, using the automated cutter such as, for example, a Sur-Size™ cutter, Model # SS-6/JS/SP, available from Azco Corp., NJ. As described herein, in one embodiment, a preferred size for the cover stock is about five inches by about three and three quarters inches (5.0″×3.75″) nonwoven piece.

Bag Making:

2. A special fixture was set up for sealing the bags. The sealing fixture was set at a temperature of two hundred ninety-six degrees Fahrenheit (296° F., 147° C.) with a dwell time of about 5.1 seconds. Air and vacuum lines should be put into place, and the targeted temperature reached to +/−two degrees Fahrenheit (2° F., 1° C.). The cover stock is then wrapped around the heated horizontal vacuum mandrel as described below.

3. A horizontal vacuum mandrel is manually rotated utilizing a hub collar until a set of double row vacuum holes are located at a predetermined location such as, for example, at a “top dead center” (e.g., a 12 o'clock position).

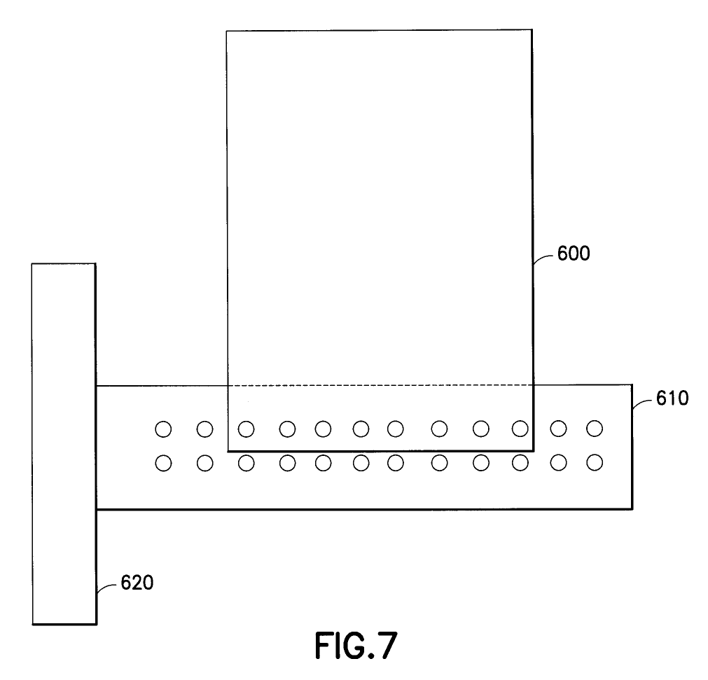

4. Place the pre-cut piece of cover stock 600 on a vacuum mandrel 610 as illustrated in FIG. 7.

5. The cover stock 600 is manually wrap around the vacuum mandrel 610 until the trailing cut edge overlaps the starting edge by about one quarter of an inch (0.25 in).

6. Grasping a hub collar 620, rotate the vacuum mandrel 610 clockwise toward the sealing bar by about ninety degrees (90°) until it clicks into place. The overlapped seam will now be facing towards the sealing bar.

7. With hands positioned clear of the mandrel 610, press the “start” button on the control panel to actuate the sealing bar.

8. After about 5.1 seconds, the sealing bar retracts and the sealed cylindrical cover stock tube is removed, by sliding it off of the mandrel.

9. After removing the cover stock cylindrical tube, the sealed overlap seam is inspected so that uniform bonding/sealing has been ensured.

10. A sufficient number of such bags are made from the cover stock pieces cut in step 1, using this special fixture.

11. Use the formed bags in the procedure described above for bagged pledgets.

Standard Procedure for Making Tampons Using the HP Simulator

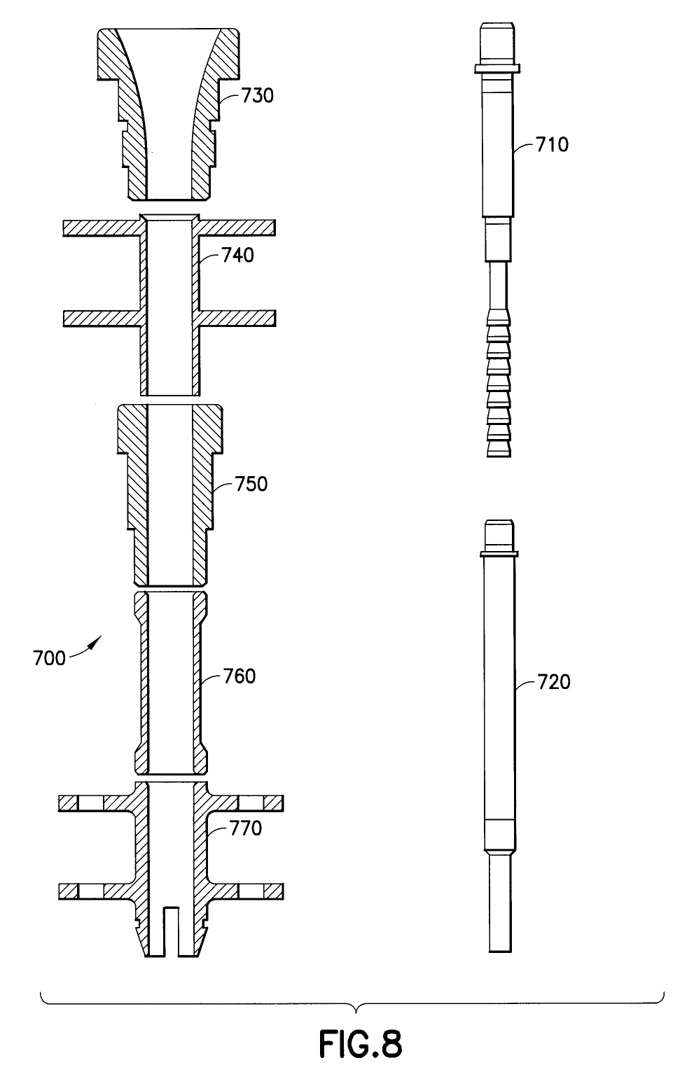

1. Install the following individual sub-component parts based on the type of pledget outlined in the test request (see instructions above). Sub-component parts include, for example, a fluted ram 710 (add shims as required), a solid ram 720 (add shims as required), a forming throat 730, a forming chain link 740, a delivery cone 750, an oven tube 760 and a stringer chain 770. FIG. 8 illustrates a detailed set up using these subcomponent parts of an HP simulator 700. More particularly, FIG. 8 illustrates the arrangement of tubes used in the formation of a folded tampon by the procedure outlined above. In the simulator 700, the fluted ram 710 is used to ram the crosspad pledget into the forming chain 740. Then, the solid ram 720 delivers the folded pledget into the heated oven tube 760, before it is ejected into the stringer tube 770 for stringing. It should be appreciated that the appropriate sizes for the various rams and tubes are selected, in accordance to what size and what absorbency range is required for the particular tampon. In one embodiment, a 0.25″ fluted ram 710 (with a 3 mm shim), a 0.374″ solid ram 720 (no shims), a 0.618″ forming throat 730, a 0.621″ forming chain 740, a 0.527″ delivery cone 750, a 0.495″ oven tube 760, and a 0.539″ stringer chain 770 were used to make the tampons described in this invention.

2. First, nonwoven webs are made by using, for example, a Rando webber (Rando Machines, N.Y.). A needle punching machine is used to form and bind the appropriate nonwoven webs together. Slitting and winding is done to form web doffs. The webs are all made in the webbing machine to target the desired web density, by adjusting the air-to-fiber ratio in the Rando machine. Typically, the web density is, for example, about 300 gsm. Then, using the automated cutting machine, as described in step 1 of the Bag Making

Instructions above, web pieces are cut to the appropriate size. For example, typically two inch by four inch (2 in.×4 in) pieces are cut.

3. Once the web pieces have been cut, place the cross-pad layup (2 web pieces or pads) on the staging platform of the simulator. The pads should be centered equally to one another to form a symmetrical cross pattern.

4. Center the lay-up under the fluted ram 710 located on the right side of the simulator 700.

5. Ensure that the forming chain 740 is positioned to the right against the mechanical stop. The forming chain 740 should be situated directly under the forming throat 730.

6. Place one finger from each hand on the left and right “Pressure Switches” simultaneously. Continue to hold these switches during the entire cycle. The machine will start, and the ram will descend as soon as both switches have pressure applied.

7. Remove both hands from the pressure switches at the end of the cycle. This is the point at which the fluted ram 710 has returned to the full up starting position.

8. With the pledget having now been inserted into the forming chain 740 and the machine stopped, the operator should swing the forming chain 740 to the left until it is against the left side mechanical stop. The forming chain 740 must now be situated directly over the delivery cone 750 and under the solid ram 720.

9. Place the appropriate size “pre-heated” oven tube 760 directly under the throat of the delivery cone 750. Engage the spring loaded oven tube retainer arm. The heated oven tube 760 should be fully inserted or the machine will jam severely during the pledget insertion cycle.

10. Once again, place one finger from each hand on the left and right “Pressure Switches” simultaneously and continue to hold switches during the entire cycle. The machine will start and ram will descend as soon as both switches have pressure applied.

11. Remove both hands from the pressure switches at the end of the cycle which will be when the solid ram 720 has returned to the full up starting position.

12. Disengage the oven tube retaining arm.

13. With a glove, remove the oven tube 760. At this point the oven tube 760 now has a formed “uncured” pledget inside.

14. Optionally, a special tapering/doming tool is used to shape the pledget and taper it to reduce the diameter at the pledget insertion end. This is done by air actuating a mandrel with a specially shaped, molded end.

15. Place the oven tube 760 with the pledget inside onto the curing oven conveyor.

16. Pledgets are then ejected out of the oven tube 760 into an appropriate sized stringer chain tube 770. Using a barbed needle, a string is attached to the pledget and then tied into a knot to secure the string to the pledget, with the needle removed. Then the pledget is removed from the stringer chain tube 770. It is then added to an appropriate size tampon applicator using an air actuated ram.

17. Finally, the applicator petals are heated to close off the applicator barrel (top portion of the applicator. This keeps the pledget from getting contaminated.

18. Steps 2 through 17 are repeated for each tampon to be made.

Syngyna Test Method (Absorbent Capacity)

Testing is done, in accordance with Standard FDA Syngyna capacity as outlined in the Federal Register Part 801, 801.43.

An un-lubricated condom, with tensile strength between 17-30 MPa, is attached to the large end of a glass chamber with a rubber band and pushed through the small end using a smooth, finished rod. The condom is pulled through until all slack is removed. The tip of the condom is cut off and the remaining end of the condom is stretched over the end of the tube and secured with a rubber band. A tampon pre-weighed (to the nearest 0.01 gram) is placed within the condom membrane so that the center of gravity of the tampon is at the center of the chamber. An infusion needle (14 gauge) is inserted through the septum created by the condom tip until it contacts the end of the tampon. The outer chamber is filled with water pumped from a temperature controlled water bath to maintain the average temperature at twenty-seven degrees Celsius (27° C.) plus or minus one (1) degree Celsius. The water returns to the water bath.

A Syngyna fluid (10 grams sodium chloride, 0.5 grams Certified Reagent Acid Fuchsin, diluted to 1,000 milliliters with distilled water) is then pumped through the infusion needle at a rate of about fifty (50) milliliters per hour. The test terminates when the tampon is saturated and the first drop of fluid exits the apparatus. The test is aborted if fluid is detected in the folds of the condom before the tampon is saturated. The water is then drained and the tampon is removed and immediately weighted to the nearest 0.01 grams. The absorbent capacity of the tampon is determined by subtracting its dry weight from the wet final weight. The condom is replaced after ten (10) tests or at the end of the day during which the condom is used in testing, whichever comes first.

Results

Table 1 below provides a list of examples conducted to illustrate aspects of the present invention. The examples include post-crosslinking of rayon fiber, specifically multilobal rayon fiber.

As can seen, several control samples were run with standard, e.g., untreated, uncrosslinked fiber for comparison purposes. The control samples were included since various nonwoven bags were used. Several examples show that hydrothermal treatments were done on fiber, using various conditions. Finally, a variety of chemically crosslinked schemes were investigated. A detailed description is provided for these examples, as well as a shorter name, for reference in subsequent data tables. The hydrothermal and chemical crosslinking schemes have been outlined above. The various treatments listed in the tables correspond to the specific schemes listed above.

TABLE 1
Description of Examples.

| | | Level of | | |
|---|---|---|---|---|
| | Crosslinking | Crosslinker or | | |
| Example | type (short | Hydrothermal | Nonwoven | |
| ID | name) | Conditions | Bag Used | Full Description |
| C1 | Control | NA | HDK | Kelheim ML Control Fiber in Bag |
| C2 | Control | NA | HDK | Kelheim ML Control Fiber in Bag |
| C3 | Control | NA | BDK | Kelheim ML Control Fiber in Bag |
| C4 | Control | NA | HDK | Kelheim ML Control Fiber in Bag |
| C5 | Control | NA | PGI-1 | Kelheim ML Control Fiber in Bag |
| C6 | Control | NA | PGI-2 | Kelheim ML Control Fiber in Bag |
| E1 | HT | 100 deg/60 min | BDK | Hydrothermal Treatment, 100 deg C., 60 minutes |
| E2 | HT | 108 deg/60 min | HDK | Hydrothermal Treatment, 108 deg C., 60 minutes |
| E3 | HT | 116 deg/45 min | PGI-1 | Hydrothermal Treatment, 116 deg C., 45 minutes |
| E4 | HT | 116 deg/45 min | BDK | Hydrothermal Treatment, 116 deg C., 45 minutes |
| E5 | HT | 116 deg/45 min | PGI-1 | Hydrothermal Treatment, 116 deg C., 45 minutes |
| E6 | HT | 116 deg/45 min | PGI-2 | Hydrothermal Treatment, 116 deg C., 45 minutes |
| E7 | HT | 116 deg/45 min | BDK | Hydrothermal Treatment, 116 deg C., 45 minutes |
| E8 | HT | 116 deg/45 min | HDK | Hydrothermal Treatment, 116 deg C., 45 minutes |
| E9 | HT | 124 deg/30 min | BDK | Hydrothermal Treatment, 124 deg C., 30 minutes |
| E10 | Cit | 1% | PGI-2 | Rayon treated with Citric acid/NaH2PO2 (1%/1%), 25% dried before curing |
| E11 | Cit | 1% | PGI-2 | Rayon treated with Citric acid/NaH2PO2 (1%/1%), 50% dried before curing |
| E12 | Cit | 1% | PGI-1 | Rayon treated with Citric acid/NaH2PO2 (1%/1%), 25% dried before curing |
| E13 | Cit | 1% | PGI-1 | Rayon treated with Citric acid/NaH2PO2 (1%/1%), 25% dried before curing |
| E14 | Gly | 1% | HDK | Rayon treated with Glyoxal/glyoxal resin/MgCl2 (1%/1%/1%), 50% dried before curing |
| E15 | Gly | 3% | HDK | Rayon treated with Glyoxal/glyoxal resin/MgCl2 (3%/3%/3%), 50% dried before curing |
| E16 | Gly | 3% | PGI-1 | Rayon treated with Glyoxal/glyoxal resin/MgCl2 (3%/3%/3%), 50% dried before curing |
| E17 | Gly | 3% | PGI-1 | Rayon treated with Glyoxal/glyoxal resin/MgCl2 (3%/3%/3%), 50% dried before curing |
| E18 | BTCA | 1% | BDK | Rayon treated with BTCA, 25% dried before curing |
| E19 | BTCA | 1% | BDK | Rayon treated with BTCA, 50% dried before curing |
| E20 | DMD | 1% | BDK | Rayon treated with DMDHEU (1%)/MgCl2 |
| E21 | DMD | 5% | BDK | Rayon treated with DMDHEU (5%)/MgCl2 |
| E22 | EDGE | 3% | PGI-2 | Rayon treated with Ethylene Glycol-Diglycidylether (EDGE) (3%) |
| E23 | EDGE | 5% | PGI-2 | Rayon treated with Ethylene Glycol-Diglycidylether (EDGE) (5%) |
| E24 | DCHTRI | 1% | HDK | Rayon treated with DCH-Triazine-NaHCO3 (1%/1%), 25% dried before curing |
| E25 | DCHTRI | 3% | HDK | Rayon treated with DCH-Triazine-NaHCO3 (3%/3%), 50% dried before curing. |

(Those labeled with C are Comparative Examples).

Table 2 provides the results for the Syngyna absorbency (absolute and gram per gram) as well as the results for the moisture values for the examples listed in Table 1 above. As shown, absorbency results are slightly lower than expected for super tampons. This is as a consequence of the bagged tampon method used to form these tampons. It should be noted that the differences in absorbency and moisture for the various treatments are quite a bit different than would be expected based upon the standard errors for these measurements. Results for Syngyna absorbency averages, for example, range from a minimum of 5.61 grams to a maximum of 9.56 grams in Table 2, even though the standard error of estimate is about 0.16 grams.

TABLE 2
Key Syngyna and Moisture Results for
the Examples Listed in Table 1

| | Cross- | | | | |
|---|---|---|---|---|---|
| | linking | Level of | | | Average |
| Ex- | type | Crosslinker or | Average | avg. g/g | Moisture |
| ample | (short | Hydrothermal | absor- | absor- | level |
| ID | name) | Conditions | bency | bency | (LOD), % |
| C1 | Control | NA | 7.825 | 2.548 | 10.360 |
| C2 | Control | NA | 7.628 | 2.489 | 10.038 |
| C3 | Control | NA | 7.364 | 2.433 | 11.960 |
| C4 | Control | NA | 7.258 | 2.402 | 11.917 |
| C5 | Control | NA | 8.036 | 2.619 | 9.833 |
| C6 | Control | NA | 8.069 | 2.634 | 10.673 |
| E1 | HT | 100/60 | 7.455 | 2.279 | 6.750 |
| E2 | HT | 108/60 | 8.704 | 2.666 | 7.063 |
| E3 | HT | 116/45 | 9.324 | 2.960 | 8.348 |
| E4 | HT | 116/45 | 7.870 | 2.474 | 8.420 |
| E5 | HT | 116/45 | 9.555 | 2.942 | 6.503 |
| E6 | HT | 116/45 | 8.899 | 2.769 | 8.225 |
| E7 | HT | 116/45 | 8.799 | 2.756 | 8.370 |
| E8 | HT | 116/45 | 8.904 | 2.701 | 6.523 |
| E9 | HT | 124/30 | 8.728 | 2.668 | 6.440 |
| E10 | Cit | 1% | 8.375 | 2.619 | 6.898 |
| E11 | Cit | 1% | 8.083 | 2.520 | 6.370 |
| E12 | Cit | 1% | 8.991 | 2.797 | 6.958 |
| E13 | Cit | 1% | 9.590 | 2.963 | 5.803 |
| E14 | Gly | 1% | 7.628 | 2.357 | 7.173 |
| E15 | Gly | 3% | 7.921 | 2.405 | 6.123 |
| E16 | Gly | 3% | 8.795 | 2.765 | 7.080 |
| E17 | Gly | 3% | 9.245 | 2.882 | 6.848 |
| E18 | BTCA | 1% | 7.579 | 2.403 | 8.923 |
| E19 | BTCA | 1% | 7.850 | 2.429 | 7.828 |
| E20 | DMD | 1% | 7.614 | 2.402 | 8.830 |
| E21 | DMD | 5% | 7.071 | 2.221 | 8.355 |
| E22 | EDGE | 3% | 8.049 | 2.523 | 6.855 |
| E23 | EDGE | 5% | 8.307 | 2.587 | 6.128 |
| E24 | DCHTRI | 1% | 6.170 | 1.876 | 5.910 |
| E25 | DCHTRI | 3% | 5.609 | 1.715 | 6.233 |

| | | | |
|---|---|---|---|
| Average standard error for | 0.156 | 0.052 | 0.229 |
| measurements (estimated from | | | |
| replicates) | | | |

Table 3 repeats some of the key data from Table 2 and provides a statistical analysis of results for some promising crosslinking treatments.

In summary, lab tests illustrate that the average absorbency results for multilobal fiber that has been heat treated in an autoclave at one hundred sixteen degrees Celsius (116° C.) for about forty-five (45) minutes (examples E3-E8) is about sixteen percent (16%) more absorbent overall, ten percent (10%) on a gram per gram basis, than that of comparable control fiber samples (C1-C6). Absorbency results may be influenced by large moisture level differences and slight forming and bagging differences. However, the inventors have noted that differences in moisture level from eight to eleven percent (8% to 11%), as reported here, are not sufficient enough to account for a sixteen percent (16%) absorbency increase. Example E3 is seen to represent a good exemplification of the inventive concepts disclosed herein.

It should be appreciated that Tables 2 and 3 illustrate that the one percent (1%) citric acid/one percent (1%) sodium hypophosphite crosslinking treatment results (e.g., examples E10-E13) also look acceptable relative to control results. These samples are even drier than those for the hydrothermal treatments, yet there is evidently a sizable (e.g., fourteen percent (14%)) absorbency increase.

The three percent (3%) glyoxal/three percent (3%) glyoxal resin/three percent (3%) magnesium chloride treatment results (e.g., examples E15-E17) also exhibit high Syngyna absorbency relative to control results. Results are about thirteen percent (13%) higher overall for this treatment. All other treatments exhibited absorbency values which were roughly comparable or statistically nearly equivalent to that of the control fiber samples. Of course, the inventors expect that slight adjustment of crosslinking conditions or levels may influence these results.

TABLE 3
Key Comparisons from Table 2: Controls vs. Hydrothermal Treatment

| | | | | | |
|---|---|---|---|---|---|
| | Avg Syngyna | Avg gram per | Avg | | |
| | Absorbency, | gram | moisture | X-linker | X-Linker |
| Example | g | absorbency | value, % | synth. type | Level/Trmt |

Control vs. Hydrothermal Treatment (116 deg C./45 min.)

| | | | | | |
|---|---|---|---|---|---|
| C1 | 7.825 | 2.548 | 10.360 | Control | NA |
| C2 | 7.628 | 2.489 | 10.038 | Control | NA |
| C3 | 7.364 | 2.433 | 11.960 | Control | NA |
| C4 | 7.258 | 2.402 | 11.917 | Control | NA |
| C5 | 8.036 | 2.619 | 9.833 | Control | NA |
| C6 | 8.069 | 2.634 | 10.673 | Control | NA |
| E3 | 9.324 | 2.960 | 8.348 | HT | 116/45 |
| E4 | 7.870 | 2.474 | 8.420 | HT | 116/45 |
| E5 | 9.555 | 2.942 | 6.503 | HT | 116/45 |
| E6 | 8.899 | 2.769 | 8.225 | HT | 116/45 |
| E7 | 8.799 | 2.756 | 8.370 | HT | 116/45 |
| E8 | 8.904 | 2.701 | 6.523 | HT | 116/45 |
| t tests | 0.0024 | 0.0182 | 0.0002 | | |
| (signif if <0.05) | | | | | |
| Avg. % difference | 15.53% | 9.77% | −28.39% | | |

Control vs. 1% Citrid Acid

| | | | | | |
|---|---|---|---|---|---|
| E10 | 8.375 | 2.619 | 6.898 | Cit | 1% |
| E11 | 8.083 | 2.520 | 6.370 | Cit | 1% |
| E12 | 8.991 | 2.797 | 6.958 | Cit | 1% |
| E13 | 9.590 | 2.963 | 5.803 | Cit | 1% |
| t tests | 0.0423 | 0.1257 | 0.0000 | | |
| (signif if <0.05) | | | | | |
| Avg. % difference | 13.81% | 8.09% | −39.73% | | |

Control vs. 3% Glyoxal

| | | | | | |
|---|---|---|---|---|---|
| E15 | 7.921 | 2.405 | 6.123 | Gly | 3% |
| E16 | 8.795 | 2.765 | 7.080 | Gly | 3% |
| E17 | 9.245 | 2.882 | 6.848 | Gly | 3% |
| t tests | 0.1195 | 0.3733 | 0.0001 | | |
| (signif if <0.05) | | | | | |
| Avg. % difference | 12.43% | 6.48% | −38.10% | | |

Although described in the context of preferred embodiments, it should be realized that a number of modifications to these teachings may occur to one skilled in the art. Accordingly, it will be understood by those skilled in the art that changes in form and details may be made therein without departing from the scope and spirit of the invention.

What is claimed is:

1. A tampon pledget, comprising:
crosslinked cellulose fibers having microstructures treated to provide improved absorbency and improved wet strength;
wherein the fibers are treated with a crosslinking agent to provide at least one of a molecular weight between crosslinks of from about 10 to about 200 and a degree of crystallinity of from about 25% to about 75%.

2. The tampon pledget of claim 1, wherein the crosslinking agent includes at least citric acid in one percent (1%) by weight based on the total weight of cellulose fibers.

3. The tampon pledget of claim 1, wherein the crosslinking agent further includes at least sodium hypophosphite in one percent (1%) by weight based on the total weight of cellulose fibers.

4. The tampon pledget of claim 1, wherein the crosslinking agent is comprised of a difunctional crosslinking agent.

5. The tampon pledget of claim 4, wherein the difunctional crosslinking agent is comprised of at least one of glyoxal and a glyoxal-derived resin.

6. The tampon pledget of claim 1, wherein the crosslinking agent is comprised of a multifunctional crosslinking agent.

7. The tampon pledget of claim 6, wherein the multifunctional crosslinking agent is comprised of a cyclic urea, glyoxal, polyol condensate.

8. The tampon pledget of claim 1, wherein the crosslinking agent is added in an amount from about a thousandth of one percent (0.001%) to about twenty percent (20%) by weight based on a total weight of cellulose fibers to be treated.

9. The tampon pledget of claim 1, wherein the crosslinking agent is added in an amount of about five percent (5%) by weight based on the total weight of cellulose fibers.

10. The tampon pledget of claim 1, wherein the cellulose fibers are derived from a raw material comprised of eucalyptus pulp and wherein an amount of time, temperature and humidity setting under which the pulp is steeped, dried, shredded, and pre-aged affects an amount of oxidative degradation and an overall average molecular weight of the fibers.

11. A method for forming crosslinked cellulose fibers, comprising
selecting a cellulose raw material;
steeping the raw material in a sodium hydroxide immersion to provide alkali cellulose;
pressing the alkali cellulose;
shredding the pressed cellulose;
aging the shredded cellulose;
reacting the aged cellulose with carbon disulphide to form cellulose xanthate;
dissolving the cellulose xanthate to form viscose;
ripening the viscose;
filtering the ripened viscose to remove undissolved materials;
degassing the filtered viscose;
spinning the degassed viscose through a spinneret to form cellulose filaments;
drawing the filaments to lengthen the cellulose chains;
purifying the drawn filaments;
cutting the purified filaments to form cellulose fibers; and
post-crosslinking by at least one of chemical or hydrothermal treatment;
wherein for a dry crosslinking formation, the method includes adding a crosslinking agent to the pressing step, and for a wet crosslinking formation, the method includes adding the crosslinking agent to at least one of the dissolving and ripening steps.

12. The method for forming of claim 11, wherein the crosslinking agent includes at least citric acid in one percent (1%) by weight based on the total weight of cellulose fibers.

13. The method of forming of claim 12, wherein the crosslinking agent further includes at least sodium hypophosphite in one percent (1%) by weight based on the total weight of cellulose fibers.

14. The method of forming of claim 11, wherein the crosslinking agent is comprised of a difunctional crosslinking agent.

15. The method of forming of claim 14, wherein the difunctional crosslinking agent is comprised of at least one of glyoxal and a glyoxal-derived resin.

16. The method of forming of claim 11, wherein the crosslinking agent is comprised of a multifunctional crosslinking agent.

17. The method of forming of claim 16, wherein the multifunctional crosslinking agent is comprised of a cyclic urea, glyoxal, polyol condensate.

18. The method of forming of claim 11, wherein the crosslinking agent is added in an amount from about a hundredth of one percent (0.001%) to about twenty percent (20%) by weight based on a total weight of cellulose fibers to be treated.

19. The method of forming of claim 11, wherein the crosslinking agent is added in an amount of about five percent (5%) by weight based on the total weight of cellulose fibers.

20. The method for forming of claim 11, further including expanding a duration of the drawing step to further lengthen cellulose chains and improve interchain hydrogen bonds to provide greater areas of crystallinity.

21. The method for forming of claim 11, wherein said post-crosslinking is by hydrothermal treatment.

22. The method for forming of claim 21, wherein said hydrothermal treatment is carried out at a temperature of about 90 to about 150 degrees Celsius.

23. The method for forming of claim 21, wherein said hydrothermal treatment is carried out at a temperature of about 100 to about 125 degrees Celsius.
